(19) United States
(12) Patent Application Publication
(10) Pub. No.: US 2026/0335151 A1
(43) Pub. Date: Oct. 29, 2026

(54) PACKAGING SYSTEM THAT PROVIDES A NEW TAMPON AND AN INTEGRATED, HYGIENIC METHOD FOR DISPOSING OF A USED TAMPON

(71) Applicant: Jaelynn Smith, Ossining, NY (US)

(72) Inventor: Jaelynn Smith, Ossining, NY (US)

(21) Appl. No.: 19/191,702

(22) Filed: Apr. 28, 2025

(51) Int. Cl. A61F 13/551

(52) U.S. Cl. CPC A61F 13/55175; A61F 13/5519; A61F 2013/55195

(57) ABSTRACT

A tampon package system is provided that includes an outer package containing both a clean tampon and a disposal bag for hygienic disposal of a used tampon. The outer package comprises a front panel, a rear panel, and an interior divider panel that defines separate compartments for the clean tampon and the disposal bag. A tab on the outer package facilitates opening to access both components. The disposal bag includes a top seal to securely close the bag after receiving a used tampon, thereby providing a sanitary, discreet, and environmentally responsible method of disposal. The system improves convenience and reduces waste associated with conventional tampon disposal practices.

### BACKGROUND OF THE INVENTION

The disposal of used feminine hygiene products, particularly tampons, remains a persistent and problematic aspect of menstruation management. While tampons are widely used due to their comfort and convenience, the options available for their disposal are limited, inconvenient, and often unsanitary. Most commercially available tampon packaging is designed solely for the sanitary storage of a clean tampon and, at best, for the disposal of the applicator. However, these packaging solutions fall short when it comes to addressing the needs of women after the tampon has been used. As a result, individuals are frequently left improvising solutions to dispose of used tampons in a manner that feels discreet and hygienic, often resorting to makeshift methods that are neither sustainable nor effective.

The predominant methods of disposal currently practiced include flushing the used tampon down the toilet or wrapping it in layers of toilet paper before discarding it in the trash. Flushing tampons is a widespread behavior, despite ongoing public awareness campaigns advising against it. This method leads to adverse environmental and infrastructural consequences, such as clogged sewage systems, costly municipal repairs, and long-term ecological harm. On the other hand, wrapping tampons in toilet paper is inefficient and wasteful, requiring significant amounts of paper to mask the appearance and absorbency of the product. Furthermore, such methods are often inadequate in containing odor or leakage, leading to discomfort and self-consciousness among users.

These limitations highlight a significant oversight in menstrual product design: the absence of an integrated, sanitary solution for the disposal of used tampons. While the market has made advancements in absorbency, comfort, and discretion in use, it has largely neglected the practical considerations and emotional discomforts associated with tampon disposal. For many women, menstruation is a routine part of life that still carries logistical burdens and social stigma—especially in shared restrooms, travel scenarios, or situations where access to sufficient toilet paper or discreet disposal methods is limited. The inability to properly dispose of used tampons without creating mess, embarrassment, or environmental harm reflects a broader gap in the design of feminine hygiene products.

Therefore, the need exists for a more practical, discreet, and hygienic method of disposing of used tampons—one that aligns with the rhythm of tampon use, requires minimal behavioral change, and eliminates the drawbacks of current disposal practices.

### SUMMARY OF THE INVENTION

The present invention relates to feminine hygiene products and, more particularly, to a packaging system that provides both a clean tampon and an integrated, hygienic method for disposing of a used tampon. The invention addresses the long-standing need for a convenient, discreet, and sanitary disposal solution that complements the routine of tampon use, without introducing significant changes to user behavior.

In one embodiment, the invention comprises an outer package that houses both a new, unused tampon and a disposal bag. The outer package includes a front panel, a rear panel, and an interior divider panel that forms separate compartments for the tampon and the disposal bag. A tab formed along the side of the outer package enables the user to open the package and retrieve both components in a simple, single action.

The disposal bag included within the package is designed to securely contain a used tampon, preventing leakage, odor, or visual exposure. The disposal bag includes a top seal to close the bag after use, enabling discreet and hygienic disposal in a trash receptacle. This configuration eliminates the need to flush tampons or wrap them in toilet paper, promoting environmentally responsible disposal while reducing user discomfort and potential embarrassment.

By integrating the new tampon and a disposal bag within the same compact package, the present invention improves the overall user experience of tampon use. It provides a seamless, all-in-one system that is practical, portable, and intuitive, fulfilling both sanitary and social needs associated with menstrual hygiene management.

These and other features, aspects and advantages of the present invention will become better understood with reference to the following drawings, description and claims.

### BRIEF DESCRIPTION OF THE DRAWINGS

FIG. 1 is a front perspective view of a preferred embodiment of the present invention;

FIG. 2 is a rear perspective view of a preferred embodiment of the present invention;

FIG. 3 is a front perspective view of a preferred embodiment of the present invention, showing the packaging in an opened position; and

FIG. 4 is a flow chart depicting steps for use of the invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

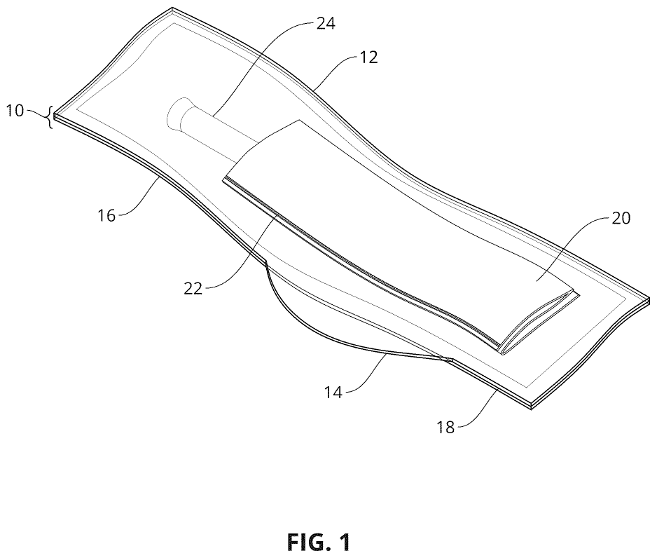

Referring now to the drawings, wherein like reference numerals are used to designate like elements throughout the several views, FIGS. 1 through 3 illustrate various views of a tampon package system designed to provide both a new tampon and an integrated disposal solution for a used tampon.

As shown in FIG. 1, the package system (10) comprises an outer package that includes a front panel (12), a rear panel (16), and an interior divider panel (18) disposed between the front and rear panels. The interior divider panel generally separates the interior of the outer package into a first compartment and a second compartment. The outer package (10) is generally rectangular in shape and is designed to be compact and portable, facilitating ease of transport in a purse, backpack, or similar container. A tab (14) is formed along one edge of the outer package (10), which allows the user to easily open the package and access its internal components within the first and second compartments.

Located within the outer package (10) is a clean tampon (24) and a disposal bag (20). The new tampon (24) is contained in one compartment of the outer package, created in part by the interior divider panel (18), which separates the tampon from the disposal bag (20) which is contained in the second compartment during storage. The disposal bag (20) is configured to receive and securely contain a used tampon. The disposal bag includes a top seal (22) that allows the bag to be closed after insertion of the used tampon, thereby preventing leakage, odor, or exposure.

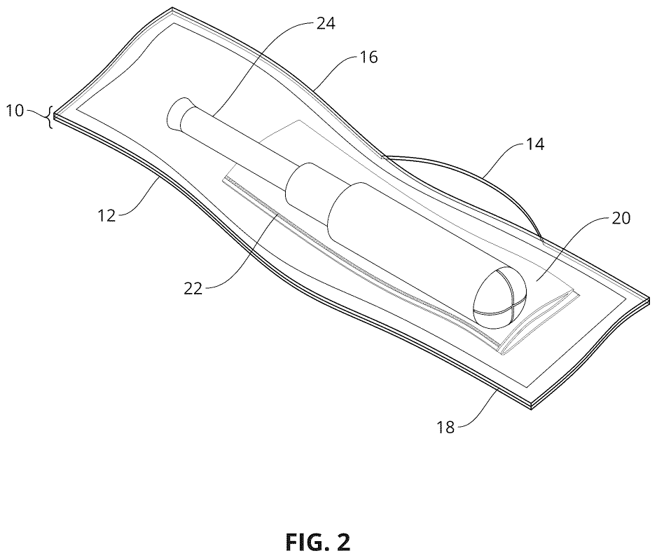

FIG. 2 further illustrates the structural arrangement of the components within the outer package (10). The front panel (12) and rear panel (16), together with the interior divider panel (18), form distinct compartments within the package. When the tab (14) is pulled, it provides access to both the new tampon (24) and the disposal bag (20), which can be removed from their respective compartments. The user may first retrieve and insert the new tampon (24), and upon removal of the used tampon, utilize the disposal bag (20) for hygienic containment.

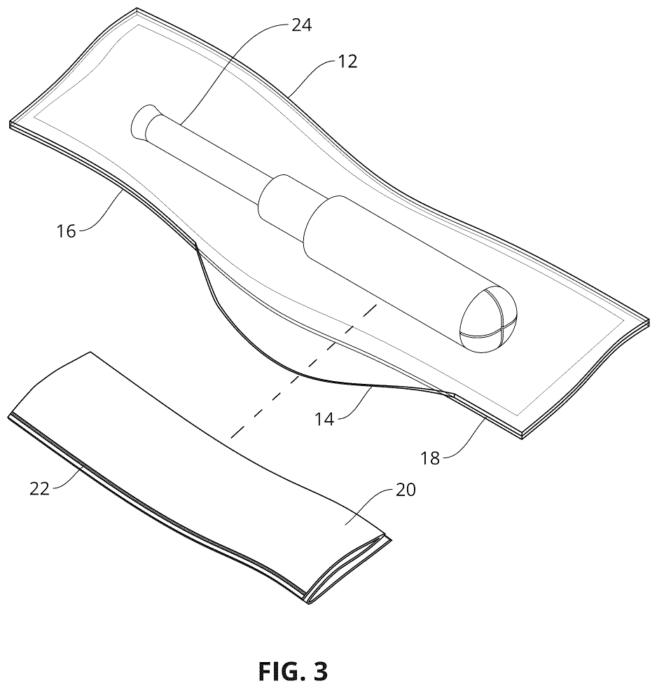

As seen in FIG. 3, the disposal bag (20) is sufficiently sized to accommodate a used tampon and is sealed at the top using the top seal (22), which may be a press-to-close zip, adhesive strip, or heat-sealed closure, to name a few. After the new tampon (24) has been used, the user may remove the disposal bag (20) from the package (10), insert the used tampon into the bag, and seal it via the top seal (22). The sealed bag may then be discreetly discarded in a trash receptacle, avoiding the need to flush or wrap the used tampon in toilet paper. This design promotes environmental responsibility and enhances the overall hygiene and convenience of tampon disposal, while maintaining user discretion and comfort.

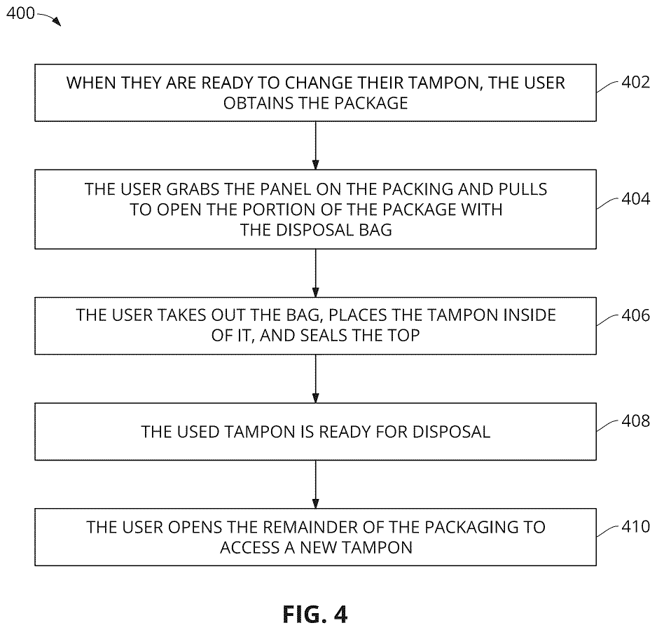

FIG. 4 depicts an exemplary flowchart (400) illustrating the steps one could use in connection with the present invention. For example, when one is ready to change their tampon, the user would gather the package of the present invention (step 402). The user can grab the panel on the package and pulls to open the package containing the new tampon and the disposal bag (step 404).

Next, the user takes out the disposal bag, places the used tampon inside the bag, and then seals the top of the bag (step 406). Once sealed in the bad, the used tampon is ready to be properly disposed, for example, in the trash can (step 408). The user can then open the remainder of the package to retrieve the new tampon (step 410).

The present invention was designed in such a way as to minimize any changes to what women are already used to. By enclosing the disposable bag to familiar tampon packaging, nothing will change in the user's routine. There is nothing extra that needs to be brought to the bathroom that women are not already used to bringing. The design of the packaging, and the steps in using the product, help maintain the barrier between the disposal bag and the new tampon until you are ready to use each. You can open the tampon section of the wrapping without having to worry about the bag falling out, and the user can take the disposal bag out and have the new tampon still completely sealed and wrapped.

It should be understood, of course, that the foregoing relates to exemplary embodiments of the invention and that modifications may be made without departing from the spirit and scope of the invention as set forth in the following claims.

What is claimed is:

1. A tampon package system comprising:
an outer package defining an interior space, the outer package including:
a front panel;
a rear panel;
an interior divider panel disposed between the front panel and the rear panel, the interior divider panel forming at least a first and a second compartment within the outer package;
a tab disposed along a side edge of the outer package, configured to open the outer package;
a new tampon disposed within the first compartment of the outer package; and
a disposal bag disposed within the second compartment of the outer package, the disposal bag comprising a top seal configured to close the disposal bag after use.

2. The tampon package system of claim 1, wherein the outer package is rectangular in shape and formed of a flexible or semi-rigid material suitable for sanitary storage and transport.

3. The tampon package system of claim 1, wherein the tab is integrally formed along a seam or edge of the front panel and the rear panel and configured to tear or peel open.

4. The tampon package system of claim 1, wherein the disposal bag is configured to receive and enclose a used tampon and prevent leakage and odor.

5. The tampon package system of claim 4, wherein the top seal of the disposal bag comprises a press-to-close zipper, an adhesive strip, or a heat-sealable material.

6. The tampon package system of claim 1, wherein the interior divider panel extends substantially the full length and width of the outer package to maintain physical separation between the new tampon and the disposal bag.

7. The tampon package system of claim 1, wherein the new tampon and the disposal bag are individually removable from the outer package through the opening created by activation of the tab.

8. The tampon package system of claim 1, wherein the outer package is configured for single use and disposable after removal of the new tampon and disposal bag.

9. The tampon package system of claim 1, wherein the new tampon is pre-positioned in the package such that it is the first component accessed upon opening the package.

10. The tampon package system of claim 1, wherein the disposal bag is folded within the second compartment in a compact form.
